(19) United States
(12) Patent Application Publication
(10) Pub. No.: US 2008/0318325 A1
(43) Pub. Date: Dec. 25, 2008

(54) Method for the Analysis of Liposomes

(75) Inventors: Gabriel Blume, Strasse (DE); Michael Sacher, Schluechtern (DE); Dirk Teichmueller, Linsengericht (DE); Katinka Jung, Berlin (DE)

Correspondence Address: MICHAEL L. DUNN, SIMPSON & SIMPSON, PLLC, 5555 MAIN STREET, WILLIAMSVILLE, NY, 14221, US

(73) Assignees: ROVI GmbH & Co. Kosmetische Rohstoffe KG, Schluechtern (DE); Gematris Test Lab GmbH, Berlin (DE)

(21) Appl. No.: 11/922,366

(22) PCT Filed: Jun. 28, 2006
(86) PCT No.: PCT/EP2006/063614

§ 371 Date: Jun. 19, 2008

(30) Foreign Application Priority Data
Jul. 1, 2005 (DE) 102005031206.3

(51) Int. Cl. G01N 33/92; G01N 24/00

(52) U.S. Cl. 436/71; 436/173

(57) ABSTRACT

A method for the determination of the morphological integrity of a membrane of lipid vesicles, such as liposomes, using electron spin resonance (ESR) spectroscopy including the steps of: a) labeling the lipid vesicles with an ESR-active probe; b) producing a sample by introducing a quantity of the labeled lipid vesicles into a test medium; c) producing a positive control by introducing a quantity of the labeled lipid vesicles into a control medium and optionally a negative control by introducing a quantity of the ESR-active probe into the test medium; d) obtaining ESR spectra of the controls and the sample; and e) comparing ESR spectra of the sample and controls to determine relative morphological integrity. Morphological integrity of lipid vesicles may be quantitatively determined in the test medium by obtaining difference spectra produced using the spectra of the sample and of the positive and/or negative control.

### BACKGROUND OF THE INVENTION

The present invention relates to a method for the qualitative and/or quantitative determination of the morphological integrity and intactness of the membrane of lipid vesicles or liposomes in a test medium, preferably a liquid test medium.

The term “liposome” stems from the Greek and means “fatty corpuscle”. Liposomes are very small hollow spheres which cannot be seen under the optical microscope and are also termed vesicles. These vesicles consist of one or more lipid double-layers which surround an aqueous core. Liposomes are at the forefront both in cosmetics and in pharmaceuticals as transport systems for active ingredients. Furthermore, in some cases, the stabilizing effect of liposomes is also exploited. Still further, liposomes themselves are also often used because of the cosmetic and pharmaceutically relevant properties of the components of the vesicles.

The first liposome to be described in the literature consisted of phospholipids (A D Bangham, Adv. Lipid Res. 1, 65-104, 1963). Even today, most liposomes consist of phospholipids, such as nanosomes. However, liposomes also encompass special liposome types such as cerasomes (ceramides), sphingosomes (sphingolipids) and niosomes (non-ionic tensides)—all “fatty corpuscles”; variations in the lipids mean that the vesicle membrane has different properties.

Usually, liposomes are produced from lecithin; normally, lecithin is obtained from the soya plant or chicken eggs. The term “lecithin” is used to describe a mixture of phospholipids (PL), oils and other lipophilic constituents or also for only the phospholipid fraction itself. In some cases, the word “lecithin” means a particular phospholipid, namely phosphatidylcholine (PC). All phospholipids consist of a lipophilic part (fatty acids) and a hydrophilic head group, wherein the fatty acids and head group are esterified via a spacer, usually glycerol.

The stability of liposomes produced from phospholipids is dependent on their phosphatidyl choline content and the composition of its fatty acids. A high phosphatidyl choline content (>70%) produces stable liposomes in aqueous formulations and in gels. The stability can be increased by adding hydrated phosphatidyl choline and/or cholesterol for the production of the vesicle.

In the cosmetics and pharmaceuticals industry, to increase the effectiveness and stability of the active substances contained in liposomes, active ingredient-loaded liposomes are incorporated into preparations. A number of different liposome preparations or formulations are commercially available in the form of sprays, gels, emulsions, lotions, creams, ointments, etc, for example. Again and again, in particular with pharmaceutical preparations, the question arises as regards stability and integrity, i.e. the morphological integrity and intactness of the vesicle in a prepared formulation. In this regard, the most important point for discussion is the possible interaction of the components of the preparation with the lipid vesicle.

Emulsifying agents and surfactants are known to solubilize lipid vesicles or liposomes. Solubilization perturbs the membrane structure of the vesicle and the morphological integrity of the membrane can no longer be guaranteed, so the advantages of vesicular encapsulation of the active ingredients are deleteriously affected or even destroyed. Such interactions between various surfactants and liposomes in aqueous solution have been described, for example, in an article by J T Simonnet (J T Simonnet, Cosmetics & Toiletries Magazine 109, 45-52, 1994).

In the cited article by Simonnet, the protective influence of various thickening agents (bio polymers) was mentioned. The various functional components of a formulation can affect the stability and thus also the integrity of the lipid vesicles/liposomes not only in a negative manner but also in a positive manner.

The importance of research and assessment of the positive and/or negative interactions of lipid vesicles/liposomes with formulation constituents is increasing with the ever-increasing demand for high-value cosmetic and pharmaceutical formulations. With regard to the effectiveness and efficiency of cosmetic and pharmaceutical formulations, it is very important to develop and establish an analytical method for assaying the stability and integrity, or morphological integrity and intactness, of lipid vesicles/liposomes in cosmetic or pharmaceutical formulations.

However, according to the prior art, quantitative determination of the stability and integrity or morphological integrity and intactness of lipid vesicles/liposomes in a cosmetic or pharmaceutical preparation is not possible or is merely unsatisfactory.

In this regard, analytical methods which could be used, such as electron microscopy (EM), static and dynamic light scattering experiments (SLS, DLS) and asymmetrical field-flow fractionation (AFFF) are only of limited application.

With EM examinations of frozen samples of cosmetic and pharmaceutical formulations, it is possible to detect lipid vesicles/liposomes and to evaluate the imaged vesicles as regards their structure (morphological integrity) and also in an ideal case their size, but that method cannot be used to quantitatively determine the quantity or fraction of undamaged and intact lipid vesicles/liposomes in a preparation. A further major disadvantage of that method lies in the fact that it is expensive both as regards procedures and apparatus. In addition, the application of EM results is strongly dependent on the experimental and interpretational experience of the researcher.

SLS and DLS experiments such as photon correlation spectroscopy (PCS) are used in the cosmetics and pharmaceuticals industry to evaluate the size and size distribution of lipid vesicles/liposomes. Those methods are of great importance as regards both product development and quality control of liposome vesicles/liposomes. However, in general, very dilute lipid vesicle/liposome emulsions are measured, rather than the preparations as they are normally used.

In SLS and DLS methods, very high dilutions of the solutions to be investigated must be used and since scattering experiments are strongly influenced and can be falsified by the presence of larger vesicles such as fat droplets from a cream formulation, those methods are not suitable for the quantitative and qualitative investigation of the morphological integrity and intactness of lipid vesicles/liposomes in cosmetic or pharmaceutical formulations.

New developments in apparatus which operate with dynamic light back scattering, for example the Horiba LB-550V (Retsch Technology GmbH, Germany) or the Zetasizer Nano Series (Malvern Instruments Ltd, Great Britain) can, albeit under optimum conditions, permit particle characterization of lipid vesicles/liposome emulsions in concentrations of 20% to a maximum of 40%. However, it is not possible to carry out investigations with undiluted samples and provide quantitative information.

Similar problems arise when using AFFF for the quantitative and qualitative examination of the morphological integrity and intactness of lipid vesicles/liposomes in cosmetic or pharmaceutical formulations. In that method, vesicles of different dimensions, for example lipid vesicles/liposomes and fat droplets in a cream formulation, can be separated and then can be independently analyzed as regards their size and size distribution. The size of the lipid vesicles/liposomes to be determined must differ substantially from that of the oil droplets in the formulation so that no overlapping of the various parameters occurs.

The AFFF method can be used to obtain quantitative information. However, here again, appropriate dilution steps must be taken when preparing the sample; they not only modify the physical properties of the cosmetic or pharmaceutical formulation, for example viscosity, rheology, transparency, etc, but they also substantially alter the physical and chemical environment of the lipid vesicles/liposomes. Thus, it can be assumed that under such conditions, the lipid vesicles/liposomes might re-organize themselves structurally. Thus, such experimental results no longer reflect the original state of the lipid vesicles/liposomes in a cosmetic or pharmaceutical preparation.

### AIM OF THE INVENTION

The invention aims to provide a method for determining the morphological integrity and intactness of lipid vesicles/liposomes in various media, in particular in cosmetic/pharmaceutical preparations, which can be used in a manner which is independent of the physical-chemical properties of the media and does not require any dilution steps.

### BRIEF DESCRIPTION OF THE DRAWINGS

FIGS. 1a to 1d show superimposed ESR spectra of sample, positive control and negative control for various test media (1a: gel, 1b: cream 1, 1c: cream 2, 1d: shampoo).

FIGS. 2a and 2b show the results of these simulations for test media cream 1 and cream 2;

FIG. 3 shows a graph of percentage contribution of intact liposomes in four different formulations over an 8 week period.

### BRIEF DESCRIPTION OF THE INVENTION

The invention is a method for the determination of the morphological integrity of a membrane of lipid vesicles, such as liposomes, using electron spin resonance (ESR) spectroscopy including the steps of:
a) labeling the lipid vesicles that are to be assayed with an ESR-active probe;
b) producing a sample by introducing a quantity of the labeled lipid vesicles into a test medium which is preferably an aqueous medium, e.g. water;
c) producing a positive control by introducing a quantity of the labeled lipid vesicles into a control medium where preferably a fraction of lipid vesicles which are morphologically undamaged is 100% in the positive control;
d) obtaining ESR spectra of the positive control and the sample; and
e) comparing ESR spectra of the sample and the positive control to determine relative morphological integrity.

The method may further include the steps of producing a negative control by introducing a quantity of the ESR-active probe into the test medium, obtaining the ESR spectra of the negative control and comparing the ERS spectra of the negative control with the ESR spectra of the sample. The ESR spectra may be conveniently recorded prior to comparing ERS spectra.

Morphological integrity of lipid vesicles may be quantitatively determined in the test medium by obtaining difference spectra produced using the spectra of the sample and of the positive control. Difference spectra may be produced using the spectra of the sample and at least one of the positive control and the spectra of the negative control.

To quantitatively determine the morphological integrity of lipid vesicles in the test medium, a simulated spectrum may be computed in which a percentage contribution of the spectrum of the positive control and a percentage contribution of the spectrum of the negative control are added to produce the simulated spectrum, wherein the percentage contributions together add up to 100%, and the simulated spectrum is compared with the spectrum of the sample. The simulated spectrum may be compared with the experimental spectrum of the sample, wherein the difference spectrum is formed by subtraction of the spectra. The percentage contributions of the positive and negative controls may be varied until the simulated spectrum substantially agrees with the experimental spectrum of the sample or until a difference spectrum produced by subtraction substantially forms a base line.

The test medium is usually selected from liquids and gels, e.g. cosmetic preparations, pharmaceutical preparations, oil-in-water emulsions, water-in-oil emulsions, hydrogels, ointments, pastes, creams and lotions.

The ESR-active probe may be a phospholipid which has a fatty acid residue substituted with a doxyl group (2,2-disubstituted 4,4-dimethyl-3-oxazolidinyloxy group) and may also be any of: 1-palmitoyl-2-(n-doxyl)-stearoyl-glycero-3-phosphocholine, wherein n=5, 7, 10, 12, 14 or 16, and preferably n=5; doxyl-5-cholesterol or a methyl ester thereof; and n-doxyl fatty acids or a methyl ester thereof, wherein preferably n=5 or 16.

### DETAILED DESCRIPTION OF THE INVENTION

The aim of the invention is achieved by dint of a method for the qualitative and/or quantitative determination of the morphological integrity and intactness of the membrane of lipid vesicles or liposomes in a test medium, preferably a liquid test medium, using electron spin resonance (ESR) spectroscopy, in which:
a) the lipid vesicles/liposomes that are to be assayed are labeled with an ESR-active sample;
b) a sample is produced by introducing a quantity of the labeled lipid vesicles/liposomes into the test medium;
c) a positive control is produced by introducing a quantity of the labeled lipid vesicles/liposomes into a control medium;
d) a negative control is produced by introducing a quantity of the ESR-active sample into the test medium;
e) ESR spectra of the sample, the positive control and the optional negative control are recorded; and
f) the recorded ESR spectra are compared with each other.

The method of the invention serves to determine the morphological integrity and intactness of membranes of lipid vesicles or liposomes in a test medium, preferably a liquid test medium.

The test medium into which the lipid vesicles/liposomes can be incorporated for research, development or for use, in particular liquid media, may be of any type. The term “liquid medium” as used in the context of the present invention encompasses low viscosity to high viscosity liquid media, including, inter alia, any cosmetic or pharmaceutical preparations such as, for example, oil-in-water and water-in-oil emulsions, hydrogels such as carbomer gels or alginate gels, and complex ointments, pastes, creams or lotions formed from many different components, including preservatives, stabilizers etc. The lipid vesicles and/or liposomes incorporated into the matrix can, for example, be present in the dissolved or embedded form, and the method of the invention can determine to what extent the vesicles dissolved, embedded or present in other manners are undamaged and intact.

The term “lipid vesicles/liposomes” as used in the context of the present invention means both double-layer membrane vesicles and multi-layer vesicles; the active ingredient may be located both in the interior of the vesicle in a solution or between the layers. Further, single-layered vesicles, termed nanoparticles, are also included.

The membrane of lipid vesicles and/or liposomes is morphologically no longer undamaged if the vesicle membrane is so affected by a medium or substance in a medium (for example emulsifiers, surfactants) that the vesicle ruptures, for example, or individual membrane constituents are released from the membrane assembly (for example by release of membrane constituents of active ingredients integrated into the membrane or membrane fragments).

A vesicle membrane is also no longer undamaged if the fluidity of the membrane is so increased that (temporary) pores are formed in the membrane, through which the medium may penetrate into the vesicle and/or material can get out from the interior of the vesicle, i.e. into the medium.

A membrane that has been affected in this manner is then no longer intact as the function of the vesicle is to separate the interior from the exterior and vice versa (vesicular encapsulation). A vesicle membrane is also no longer intact if the membrane appears undamaged from outside because the vesicle neither been ruptured nor can fragments or pores be seen, and despite the apparent integrity, the fluidity of the membrane has been increased so much that the barrier properties of the membrane have been affected so much that media or active ingredient molecules can move through the apparently undamaged membrane, or individual membrane or active ingredient molecules can be released from the membrane.

The present invention provides a method by which the intactness and integrity of vesicle membranes can be determined using electron spin resonance (ESR) spectroscopy.

Electron spin resonance (ESR) is based on the absorption of microwaves by a paramagnetic sample which is orientated in a magnetic field. It is a suitable spectroscopic method for the investigation of physico-chemical processes in biological membranes and artificial membranes. Spin labeling enables the membrane's properties to be characterized, such as fluidity and mobility, and allows interactions between lipophilic molecules and the membrane lipids to be characterized. To this end, the sample is labeled paramagnetically and then ESR-active probes are used, which are usually represent analogous lipid molecules in the membrane to be investigated. This mainly means fatty acids, esters, phospholipids, cholesterols and derivatives thereof, which are provided with a paramagnetic ESR-active group such as a doxyl group (2,2-disubstituted 4,4-dimethyl-3-oxazolidinyloxyl group). Some of said ESR probes are commercially available. An example of an ESR-active probe for use in the invention is 1-palmitoyl-2-(n-doxyl)-stearoyl-glycero-3-phosphocholine, in which n=5, 7, 10, 12, 14 or 16; preferably, n=5. Alternatively, ESR-active probes which are suitable for carrying out the method of the invention are 5-doxyl cholesterol or a methyl ester thereof and n-doxyl fatty acids or a methyl ester thereof, in which n is preferably 5 or 16.

Spin labeling can be added to the membranes in the form of ESR-active probes using various methods. When using artificial membranes, the spin labeling can, for example, be added to the lipids before the membrane is produced (pre-labeling). In this manner, the distribution of the spin labeled molecules is immediately homogeneous within the membrane. Alternatively, membranes can also be post-labeled, either by adding the ESR-active probe to a cell or liposome suspension in a suitable solution, or by slow take up of the ESR-active probe as a solid by dissolution (post-labeling). In the case of post-labeling, very low concentrations of ESR-active probes must be used so that a homogeneous distribution of the probe in the membrane can be set up.

A lipid membrane is an ordered, fluid system in which the molecules of the ESR-active probe only have a limited degree of freedom as regards their mobility. This is reflected in the high anisotropy of the spectral profile, which thus results in a structured ESR signal. Any disturbances in the ordered system of the lipid membrane changes the orientation, the environment and the mobility of the ESR-active probe which manifests itself in the form of changes in the ESR signal.

The anisotropic freedom of movement of the ESR-active probe in a membrane can be described as the order parameter S₃₃, which can take a value between 0 and 1. The higher the order parameter S, the more arranged and rigid is the membrane. The fluidity of a membrane is described by the correlation time τ_{R}. The correlation time is the time taken by the ESR-active probe to turn about its own longitudinal axis. In membranes, τ_{R} is about 10⁻⁸-10⁻⁹ seconds. The shorter the correlation time, the more fluid is the membrane.

A simulation of the spectral profile via the quantum mechanical parameters allows the order parameter S₃₃ and the correlation time τ_{R} of the ESR-active probe in a particular membrane to be quantified. This type of evaluation requires suitable simulation or fitting programs (NNSL, Freed & Schneider).

However, the mobility parameters are not of primary importance to the method of the present invention. Moreover, the spectral profile should be able to show how many lipid vesicles/liposomes present in a particular test medium, for example in a particular cosmetic/pharmaceutical formulation, are undamaged and intact and what is the fraction of lipid vesicles/liposomes for which this is no longer the case.

In the method of the invention, the lipid vesicles/liposomes to be investigated are initially labeled with the ESR-active probe. Next, the labeled lipid vesicles/liposomes are incorporated into various media, namely into at least one control medium for a positive control and into a test medium, the influence of which on the morphological integrity and intactness of the lipid vesicles/liposomes is to be investigated.

Using the labeled lipid vesicles/liposomes, the liposomal system per se is initially characterized and an ESR spectrum of a positive control (positive spectrum) is defined, whereby it is assumed that in positive control, 100% of the lipid vesicles/liposomes are intact. To produce the positive control, a quantity of the labeled lipid vesicles/liposomes is added to the appropriate control medium. Preferably, the control medium is an aqueous solution, and particularly preferably pure water as water is known not to destroy or disturb the lipid vesicles/liposomes but to stabilize it, and it can thus be assumed that the lipid vesicles/liposomes in an aqueous emulsions is 100% stable, i.e. intact.

In a further step, the lipid vesicles/liposomes of interest are incorporated into the test medium to be investigated. As already mentioned, the test medium can be a gel, a cream, an ointment or any other cosmetic or pharmaceutical formulation into which the lipid vesicles/liposomes of interest can be incorporated. An ESR spectrum (sample spectrum) is then made of the sample obtained.

Further, for particular implementations of the method of the invention, a negative control may optionally be produced in which a quantity of ESR-active probe is incorporated into the test medium to be examined; an ESR spectrum of the negative control is also recorded. Since the negative control contains no lipid vesicles/liposomes but only free mobile probe, it corresponds to 0% intact or undamaged lipid vesicles/liposomes in the test medium.

To determine the morphological integrity and intactness of the membrane of the lipid vesicles/liposomes in the test medium, the ESR spectra are compared with each other. Depending on the comparison method used, a qualitative or quantitative determination of the integrity and intactness is possible.

A qualitative determination determines whether the sample spectrum corresponds completely or substantially completely with the positive spectrum or whether it differs widely from it. With substantial correspondence, it can be assumed that all or nearly all of the lipid vesicles/liposomes in the test medium are intact. In some cases this is sufficient to assess whether the test medium is suitable for the lipid vesicle/liposome used, in particular in cases in which a clear agreement exists between the sample spectrum and the positive spectrum.

Optionally, with qualitative determination it is also possible to examine whether the sample spectrum corresponds completely or substantially completely with the negative spectrum or whether it differs widely from it. If they correspond, it can be assumed that none of or almost none of the lipid vesicles/liposomes in the test medium are intact. This is often sufficient to confirm that the test medium is unsuitable for the lipid vesicles/liposomes used.

With quantitative determination, the percentage of the undamaged or intact lipid vesicles/liposomes in the matrix is determined or calculated as follows:

The vesicles are entirely intact if the positive spectrum is not significantly different from the spectrum of the lipid vesicles/liposomes in the test medium. To determine this, the positive spectrum and sample spectrum are subtracted from each other. If the two spectra do not differ significantly from each other (approx 100% intactness), the result of this subtraction is a base line which has zero intensity apart from background noise.

The vesicles are completely non-intact, i.e. 0% intactness, if the negative spectrum is not significantly different from the spectrum of the lipid vesicles/liposomes in the test medium to be investigated. For this determination, the negative spectrum and sample spectrum are subtracted from each other. If the two spectra do not differ substantially from each other (approx 0% intactness), the result of this subtraction is a base line which has zero intensity apart from background noise.

If neither the positive nor the negative spectra correlation produce a base line which has zero intensity apart from background noise, i.e. both the positive and the negative controls differ significantly from the sample spectrum, then in accordance with the invention, for quantification, i.e. to determine the fraction of undamaged and intact or not undamaged and non-intact lipid vesicles/liposomes, a simulated spectrum is produced or calculated.

The simulated spectrum is composed of various percentages of the spectra of the positive control and the negative control, wherein the percentage of the spectrum of the positive control and the negative control are varied in such a manner that the simulated spectrum gradually iterates towards the experimental sample spectrum. When the significance of the differences between the simulated spectrum and the experimental sample spectrum is below a p of 0.05, the simulation is assumed to be acceptable. The percentage contribution of the spectrum of the positive control to the simulated spectrum then provides the percentage of morphologically undamaged or intact lipid vesicles/liposomes in the test medium. In contrast, the percentage contribution of the negative spectrum to the simulated spectrum represents the percentage of non undamaged or non-intact lipid vesicles/liposomes in the test medium.

Clearly, in the method of the invention, the determinations can be made using reference samples which contain a known quantity of intact and non-intact lipid valves/liposomes. A comparison of the ESR spectrum of the reference which has a known quantity of intact and non-intact lipid vesicles/liposomes (for example 75% intact, 25% non-intact lipid vesicles/liposomes) with that of the sample provides information as to whether the intactness of the lipid vesicles/liposomes in the sample is close to that of the reference and is still sufficient for use in a formulation, or whether it goes beyond the reference. When using a plurality of reference samples which have varying quantities of intact and non-intact lipid vesicles/liposomes in control media (for example 90%:10%, 80%:20%, 70%:30% intact: non-intact lipid vesicles/liposomes), semiquantitative determinations can be made.

It will be clear to the person skilled in ESR spectroscopy that to compare and evaluate the various ESR spectra for the sample and the positive and negative controls, it is important to adjust the measurement conditions so that the spectra have a comparable or identical signal-to-noise ratio. For this purpose, appropriate preliminary tests are advantageously carried out in which various concentration ratios and quantities of ESR-active probes, lipid vesicles/liposomes and/or media are produced and the ESR measurement parameters are altered to obtain identical signal-to-noise ratios. Lower concentrations of ESR-active probes can, for example, be balanced out by higher amplification and larger accumulations. With this method, it is possible to check that the ESR-active probe does not diffuse out of the liposomes. Further, the maximum quantity of liposomes which can be incorporated into a cosmetic formulation can be defined. Furthermore, it is advantageous to standardize the spectra to a unitary integral value used to determine the integrals of the individual spectra and to normalize the spectra.

Further features and possible combinations of features and the advantages resulting from the further features and possible combinations of features of the present invention will be illustrated using the examples below and the accompanying figures.

### EXAMPLES

A—Labeling of Liposomes

During preliminary tests, phosphatidyl choline liposomes were labeled with ESR-active probe using a post-labeling method; various molar ratios of probes and membrane lipids of the corresponding vehicle system were produced and measured until an optimum concentration of the probe was found which produced stable spectra with an acceptable signal-to-noise ratio.

The ESR-active probe for spin labeling was 16:0-05 PC DOXYL (1-palmitoyl-2-stearoyl-(5-doxyl)-sn-glycero-3-phosphocholine; Avanti Polar Lipids, Inc, Alabaster, Ala., USA). Firstly, a solution with a concentration of 4.6 mM of probe in 96% ethanol was produced. Aliquots of this solution were added to the liposomes under investigation so that the final concentration of ESR-active probe in the liposome suspension was 0.2 mM.

B—Production of Samples Positive Controls and Negative Controls

For the samples, labeled liposomes in a concentration of 5% by weight were added to the test media to be investigated. For the positive controls, the labeled liposomes were suspended in water in a concentration of 5% by weight. For the negative controls, 10 μM of ESR-active probe was incorporated into the various test media. The samples were homogenized by continuous stirring followed by brief centrifuging steps at 350 g for 6 seconds.

The final concentration of the ESR-active probes in all samples was 10 μM.

C—Measurement of Spectra

The ESR spectra of the labeled liposomes in the samples and the positive controls were recorded (i) directly after stirring in the liposomes (t=0), (ii) after 24 h at room temperature (RT), (iii) after 24 h at RT plus 8 h at 40° C., (iv) after 14 days at RT, (v) after 4 weeks at RT and (vi) after 8 weeks at RT.

For the measurements, the samples were sucked into a glass capillary pipette (50 μl), the capillary pipette was sealed with hematocrit wax and the ESR spectrum was recorded. All spectra were recorded using the same apparatus and the same measurement parameters:

Apparatus: ESR MS200 (Magnetech, Berlin);

Measurement parameters: 3360 G mean field, 100 G scan width, 10 mW attenuation, 20 sec sweep, 300 amplification, 1 G modulation amplitude, 40 accumulations.

D—Qualitative Comparison of Spectra

To determine whether the positive spectrum differed significantly from the spectrum of liposomes in the corresponding test medium, the positive spectrum and sample spectrum were subtracted from each other. The result of this subtraction produced a base line when the two spectra did not significantly differ from each other.

E—Quantitative Comparison of Spectra

For the quantitative determination of liposome stability, the spectra were normalized, i.e. the individual spectra were divided by the intensity of the absorption spectrum. For each evaluation, the positive spectrum, the negative spectrum and the experimental sample spectrum (labeled liposomes in a formulation) were superimposed. If a heterogeneous situation occurred in the sample, i.e. if part of the liposomes in the sample were intact and another part non-intact, the corresponding sample spectrum deviated from both the positive and from the negative spectrum. By summing the percentage contributions of the signals of the positive spectrum and the negative spectrum, a calculated (simulated) spectrum was produced which, by varying the corresponding percentage contributions, was iterated towards the sample spectrum. The best possible iterated simulated spectrum was then subtracted from the experimental spectrum. When the significance of the difference in the individual signal regions was under p=0.05, it was assumed that the simulation was significantly representative of the percentage contribution of the intact and non-intact liposomes in the sample.

F—Results

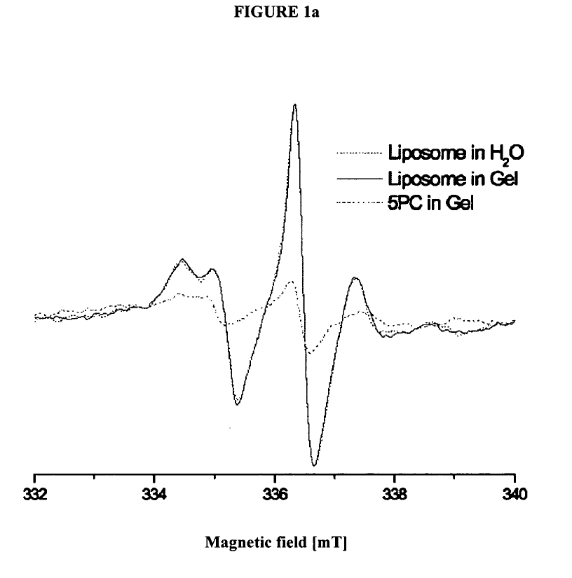

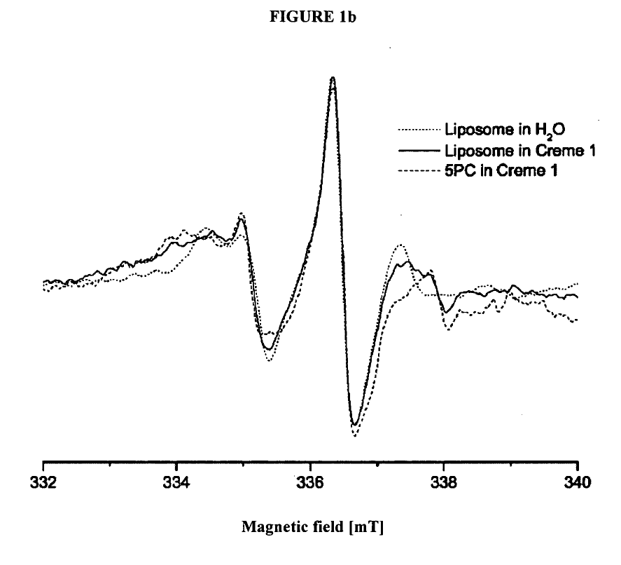

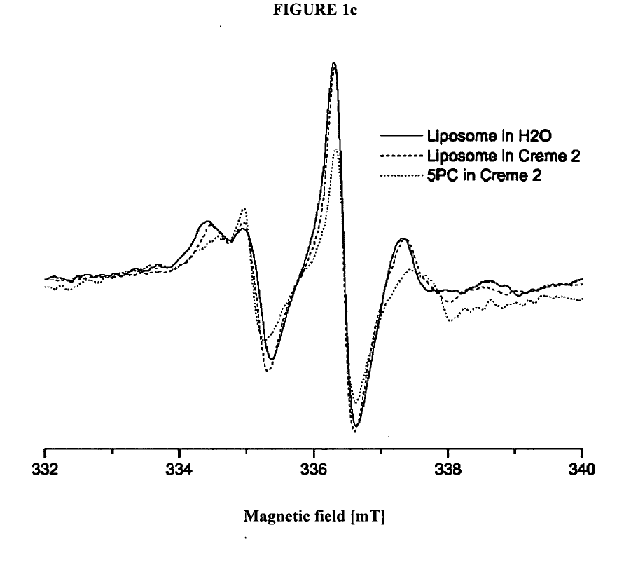

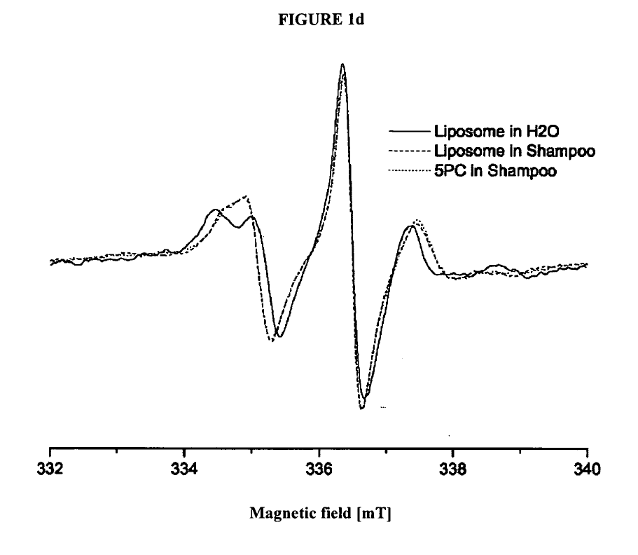

FIGS. 1a to 1d show superimposed ESR spectra of sample, positive control and negative control for various test media (1a: gel, 1b: cream 1, 1c: cream 2, 1d: shampoo). The ESR spectra were recorded as described above.

FIG. 1a shows a spectrum of the liposomes in the gel, wherein the spectrum does not significantly differ from the positive spectrum (liposomes in H₂O). This is proof that the liposomes in the gel were undamaged and intact. Similarly, the spectrum of the liposomes in the gel differed substantially from the negative spectrum of the free ESR-active probe in the gel (16:0-05 PC DOXYL in gel).

FIG. 1d shows an example of a test medium, namely shampoo, in which the liposomes are unstable, i.e. not undamaged and not intact. Similarly, the spectrum of the liposomes in the gel did not differ substantially from the negative spectrum (16:0-05 PC DOXYL in gel).

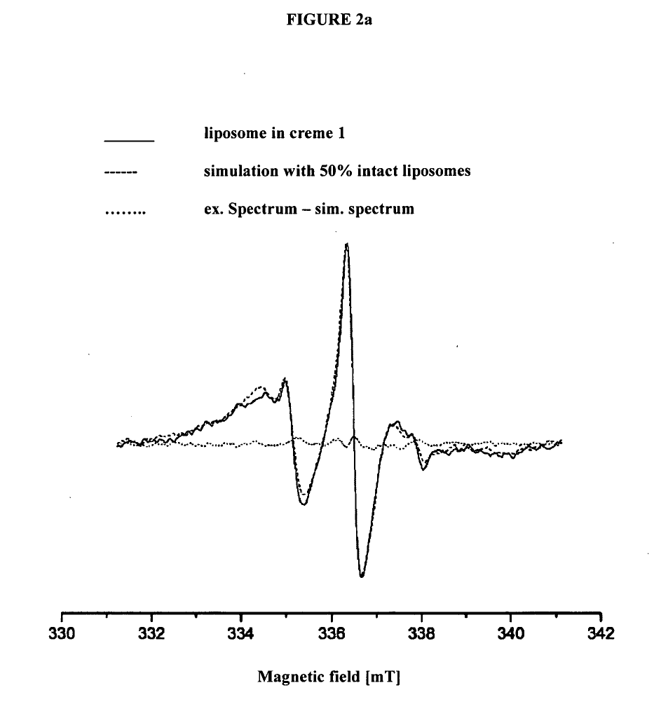

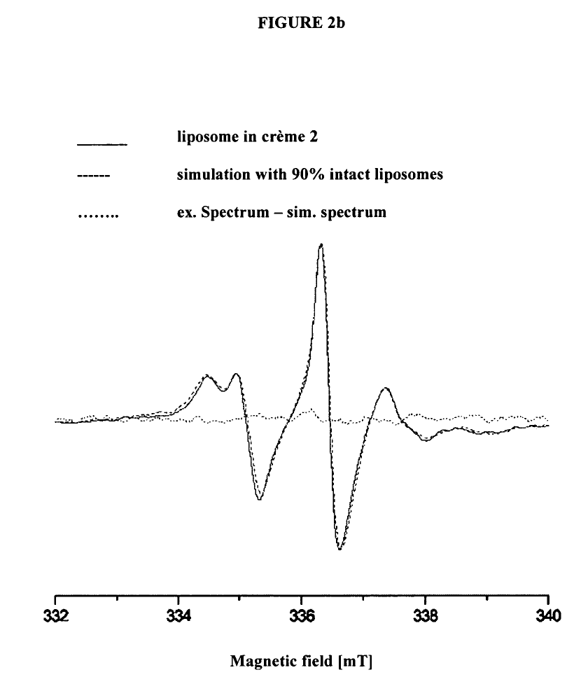

FIGS. 1b (cream 1) and 1c (cream 2) show intermediate situations, in which the spectra of the liposomes in the creams differ significantly from the positive spectrum and from the corresponding negative spectrum. Thus, to quantitatively determine the percentage of intact and non-intact liposomes in the probes, different percentages of positive and negative spectra were summed until the resulting simulated spectrum did not substantially differ from the experimental spectrum. FIGS. 2a and 2b show the results of these simulations for the test media cream 1 and cream 2.

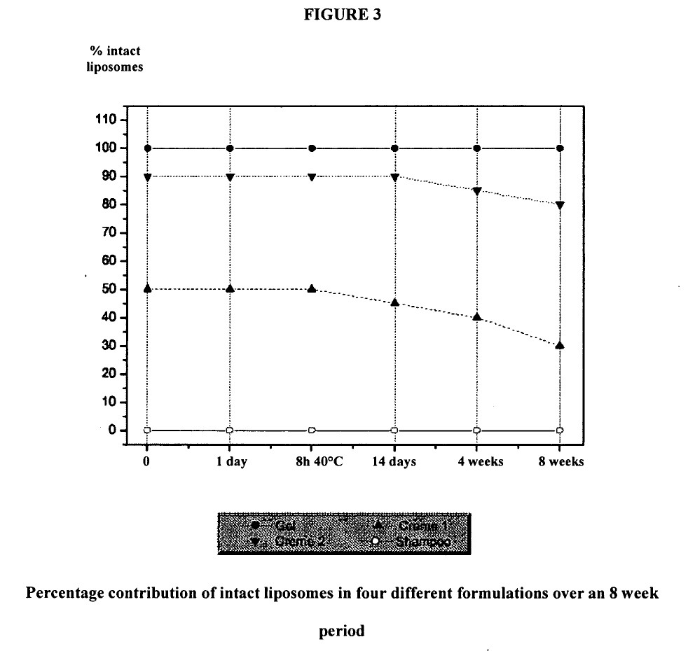

This method was carried out on the spectra of the liposomes in all test media (i) immediately after stirring in the liposome (t=0), (ii) after 24 h at room temperature (RT), (iii) after 24 h at RT plus 8 h at 40° C., (iv) after 14 days, (v) after 4 weeks and (vi) after 8 weeks. The results are summarized in Table 1 and shown graphically in FIG. 3.

TABLE 1
Percentage of intact liposomes in four different formulations over a period.

| | % intact liposomes | | | | |
|---|---|---|---|---|---|
| | | After 24 h + | | | |
| Formulation | t = 0 | 8 h 40° C. | After 14 days | After 4 weeks | After 8 weeks |
| Gel¹⁾ | 100% | 100% | 100% | 100% | 100% |
| Cream 1²⁾ | 50% | 50% | 45% | 40% | 30% |
| Shampoo³⁾ | 0% | 0% | 0% | 0% | 0% |
| Cream 2⁴⁾ | 90% | 90% | 90% | 85% | 80% |

¹⁾Gel formulation with following INCI composition: water, sorbeth-30, polysorbate, carbomer, preservative;
²⁾Commercial cream formulation;
³⁾Commercial shampoo formulation;
⁴⁾Cream formulation with following INCI composition: water, hydrated jojoba oil, steareth-2, glycerin, PPG-15, stearyl ether, hydrated canola oil, dioctyladipate, steareth-21, dicaprylyl ether, shea butter, cyclomethicone, polyacrylamide, C13-14-isoparaffin, laureth-7, xanthan, sodium hyaluronate, preservative.

What is claimed is:

1-10. (canceled)

11. A method for the determination of the morphological integrity of a membrane of lipid vesicles using electron spin resonance (ESR) spectroscopy, including the steps of:
a) labeling the lipid vesicles that are to be assayed with an ESR-active probe;
b) producing a sample by introducing a quantity of the labeled lipid vesicles into a test medium;
c) producing a positive control by introducing a quantity of the labeled lipid vesicles into a control medium;
d) obtaining ESR spectra of the positive control and the sample; and
e) comparing ESR spectra of the sample and the positive control to determine relative morphological integrity.

12. The method of claim 1 where the vesicle is a liposome.

13. The method of claim 1 including the steps of producing a negative control by introducing a quantity of the ESR-active probe into the test medium, obtaining the ESR spectra of the negative control and comparing the ERS spectra of the negative control with the ESR spectra of the sample.

14. The method of claim 1 where prior to step e), the ESR spectra are recorded.

15. The method of claim 13 where prior to comparing the ERS spectra of the negative control with the ESR spectra of the sample, the ESR spectra of the negative control are recorded.

16. A method according to claim 11 wherein the ESR-active probe is a phospholipid which has a fatty acid residue substituted with a doxyl group (2,2-disubstituted 4,4-dimethyl-3-oxazolidinyloxy group).

17. A method according to claim 11 where the ESR-active probe is selected from the group consisting of:
1-palmitoyl-2-(n-doxyl)-stearoyl-glycero-3-phosphocholine, doxyl-5-cholesterol or a methyl ester thereof; and n-doxyl fatty acids or a methyl ester thereof.

18. A method according to claim 11 where a fraction of lipid vesicles which are morphologically undamaged is 100% in the positive control.

19. A method according to one of claims 11 wherein the medium for the positive control is an aqueous medium.

20. A method according to claim 11 wherein to quantitatively determine the morphological integrity of lipid vesicles in the test medium, difference spectra are produced using the spectra of the sample and of the positive control.

21. A method according to claim 13 wherein to quantitatively determine the morphological integrity of lipid vesicles in the test medium, difference spectra are produced using the spectra of the sample and at least one of the positive control and the spectra of the negative control.

22. A method according to claim 13 in which, to quantitatively determine the morphological integrity of lipid vesicles in the test medium, a simulated spectrum is computed in which a percentage contribution of the spectrum of the positive control and a percentage contribution of the spectrum of the negative control are added to produce the simulated spectrum, wherein the percentage contributions together add up to 100%, and the simulated spectrum is compared with the spectrum of the sample.

23. A method according to claim 22, in which the simulated spectrum is compared with the experimental spectrum of the sample, wherein the difference spectrum is formed by subtraction of the spectra.

24. A method according to claim 22 in which the percentage contributions are varied until the simulated spectrum substantially agrees with the experimental spectrum of the sample.

25. A method according to claim 22 in which the percentage contributions are varied until a difference spectrum produced by subtraction substantially forms a base line.

26. A method according to claim 11 in which the test medium is selected from liquids and gels selected from the group consisting of cosmetic preparations, pharmaceutical preparations, oil-in-water emulsions, water-in-oil emulsions, hydrogels, ointments, pastes, creams and lotions.

27. A method according to claim 17 where n=5, 7, 10, 12, 14 or 16 in the 1-palmitoyl-2-(n-doxyl)-stearoyl-glycero-3-phosphocholine and wherein n is 5 or 16 in the n-doxyl fatty acids or a methyl ester thereof.
